(19) United States
(12) Patent Application Publication
(10) Pub. No.: US 2020/0299625 A1
(43) Pub. Date: Sep. 24, 2020

(54) BIOCOMPATIBLE SURFACE COATING WITH HIGH SURFACE WETTABILITY PROPERTIES

(71) Applicant: Corning Incorporated, Corning, NY (US)

(72) Inventor: Vasiliy Nikolaevich Goral, Painted Post, NY (US)

(21) Appl. No.: 16/089,459

(22) PCT Filed: Apr. 11, 2017
(86) PCT No.: PCT/US17/26883

§ 371 Date: Sep. 28, 2018

Related U.S. Application Data
(60) Provisional application No. 62/322,015, filed on Apr. 13, 2016.
(60) Provisional application No. 62/395,575, filed on Sep. 16, 2016.

(51) Int. Cl. C12M 1/00

(52) U.S. Cl. CPC C12M 23/20

(57) ABSTRACT

A device (e.g., 3D cell culture device, 2D cell culture device, microfluidic device) is described herein which has a dissolvable carbohydrate coating with high surface wettability properties. In addition, methods for using and manufacturing the device are described herein.

This application claims the benefit of priority under 35 U.S.C. § 119 of U.S. Provisional Application Ser. No. 62/322,015 filed on Apr. 13, 2016 and 62/395,575 filed on Sep. 16, 2016, the contents of which are relied upon and incorporated herein by reference in their entirety.

### TECHNICAL FIELD

The present disclosure relates to a device (e.g., 3D cell culture device, 2D cell culture device, microfluidic device) which has a dissolvable surface coating with high surface wettability properties. The present disclosure also relates to methods for using and manufacturing the device which has the dissolvable surface coating with high surface wettability properties.

### BACKGROUND

It is well known that devices such as, for example, 3D cell culture devices, 2D cell culture devices, and microfluidic devices are critical for enabling research in molecular, cellular, and biological fields to name a few. For example, 3D cell culture devices which generate 3D cellular aggregates (e.g., multicellular spheroids) have been recognized over the past decade to be an important part of fundamental research as well as cell therapy. Currently, the advantages of using 3D cell culture devices for both basic research and therapeutic development before having to perform animal studies is a driving force for the continual development and commercialization of new 3D cell culture products which are capable of generating and supporting 3D cellular aggregates (e.g., multicellular spheroids).

The 3D cell culture devices typically have a surface which hinders cells from attaching to the surface which in turn induces the formation of 3D cellular aggregates (e.g., multicellular spheroids). One of the most successful methods to generate cellular 3D aggregates is to seed cells in wells with a non-adherent cell culture surface. An example of a line of products which have wells with a non-adherent cell culture surface referred to as an ultra low attachment (ULA) surface are sold by Corning Incorporated. These products generally work very well but can still be improved upon so as to be able to better generate cellular 3D aggregates of uniform sizes.

Another method used today to generate cellular 3D aggregates is to seed cells on surfaces or in wells which have microcavities to contain 3D aggregates or spheroids. An example of 3D cell culture devices that has wells with microcavities surfaces that can be used to generate 3D cellular aggregates are AggreWell™ Plates which are sold by StemCell Technologies. The AggreWell™ line of products is represented in multiwell format with a 6-well plate being the largest. However, because of the acute angels of the microwell or microcavity geometry these large footprint products are susceptible to air bubble entrapment during the initial cell seeding step. One way that StemCell Technologies recommends to address this air bubble entrapment problem, is to fill the microwells completely so that the plates can be centrifuged with media before adding the cells. However, this proposed solution is undesirable in cell culture protocol because it significantly limits the user's ability to handle multiple plates simultaneously. Essentially, the air bubble entrapment in microwell cavities or microcavities during the initial cell seeding step poses significant problems for the adoption and design of large footprint 3D cell culture devices which can be used to generate bulk quantities of 3D cell aggregates. Hence, the products which have wells with microcavities can be improved upon so as to address the air bubble entrapment problem in order to be able to better generate cellular 3D aggregates.

In view of at least the foregoing, it can be appreciated that improvements are desirable for 3D cell culture devices. In fact, improvements are desirable for a wide range of devices in addition to the 3D cell culture devices including, for example, 2D cell culture devices and microfluidic devices. The present disclosure addresses these needs and other needs.

### SUMMARY

A device (e.g., 3D cell culture device, 2D cell culture device, microfluidic device) and methods for manufacturing and using the device which addresses the aforementioned needs are described in the claims of the present application. Advantageous embodiments of the device and the methods for manufacturing and using the device are also described in the claims.

In one aspect, the present disclosure provides a device (e.g., 3D cell culture device, 2D cell culture device, microfluidic device) which has a polymer surface with a dissolvable carbohydrate coating (i.e., a biocompatible surface coating with high surface wettability properties) located thereon. The polymer surface can be one of the following (for example): ethylene vinyl acetate, polypropylene, polyolefin, polystyrene, plasma-treated polystyrene, polycarbonate, polyester, copolymers of polyester, and a fluoropolymer. The water dissolvable carbohydrate coating can be made from one of the following (for example) monosaccharides, disaccharides, oligosaccharides, and polysaccharides. In embodiments, the dissolvable carbohydrate coating can contain polyethylene oxide or like compound to stabilize the resulting water dissolvable carbohydrate coating for long term storage at ambient conditions. If desired, the polymer surface may be coated with a low protein binding coating (e.g., ULA coating) which in turn is coated with the water dissolvable carbohydrate coating. The device is a marked improvement over the state-of-the-art devices because it has a dissolvable carbohydrate thin coating which not only increases the surface wettability properties but also does not impact the physical-chemical properties of the polymer surface because the carbohydrate thin coating dissolves upon the addition of a water based cell culture media.

In another aspect, the present disclosure provides a method for manufacturing a device (e.g., 3D cell culture device, 2D cell culture device, microfluidic device) which comprises the steps of (a) providing a polymer surface; and (b) applying a water dissolvable carbohydrate coating (i.e., a biocompatible surface coating with high surface wettability properties) onto the polymer surface. The applying step can entail a spray coating step, a solvent coating step, or a spin coating step. For example, the applying step can comprise the steps of bringing a solution of carbohydrate and solvent into direct contact with the polymer surface, and evaporating the solvent from the solution resulting in the polymer surface being coated with the water dissolvable carbohydrate coating that is not chemically bound to the polymer surface. In one embodiment, the polymer surface may have a low protein binding coating (e.g., covalently bound hydrogel layer) on which there is applied the water dissolvable carbohydrate coating. The polymer surface can be one of the following (for example): ethylene vinyl acetate, polypropylene, polyolefin, polystyrene (e.g., plasma-treated polystyrene), polycarbonate, polyester, copolymers of polyester, and a fluoropolymer. The water dissolvable carbohydrate coating can be made from one of the following (for example) monosaccharides, disaccharides, oligosaccharides, and polysaccharides. The dissolvable carbohydrate coating can contain polyethylene oxide or like compound to stabilize the resulting water dissolvable carbohydrate coating for long term storage at ambient conditions. The device is a marked improvement over the state-of-the-art devices because it has a water dissolvable carbohydrate thin coating which not only increases the surface wettability properties but also does not impact the physical-chemical properties of the polymer surface because the carbohydrate thin coating dissolves upon the addition of a water based cell culture media.

In yet another aspect, the present disclosure provides a method for using a device (e.g., 3D cell culture device, 2D cell culture device, microfluidic device) which comprises the steps of (a) providing the device which has a polymer surface having a dissolvable carbohydrate coating (i.e., a biocompatible surface coating with high surface wettability properties) located thereon, and (b) adding a cell media solution which contains at least water, a carbohydrate, and cells onto the dissolvable carbohydrate coating which is located on the polymer surface. In one embodiment, the polymer surface may have a low protein binding coating (e.g., covalently bound hydrogel layer) on which there is located the water dissolvable carbohydrate coating. The polymer surface can be one of the following (for example): ethylene vinyl acetate, polypropylene, polyolefin, polystyrene (e.g., plasma-treated polystyrene), polycarbonate, polyester, copolymers of polyester, and a fluoropolymer. The dissolvable carbohydrate coating can be made from one of the following (for example) monosaccharides, disaccharides, oligosaccharides, and polysaccharides. The dissolvable carbohydrate coating can contain polyethylene oxide or like compound to stabilize the resulting dissolvable carbohydrate coating for long term storage at ambient conditions. The device is a marked improvement over the state-of-the-art devices because it has a dissolvable carbohydrate coating which not only increases the surface wettability properties but also does not impact the physical-chemical properties of the polymer surface because the carbohydrate thin coating dissolves upon the addition of a water based cell culture media.

Additional aspects of the disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

A more complete understanding of the present disclosure may be had by reference to the following detailed description when taken in conjunction with the accompanying drawings wherein:

FIGS. 1A and 1B are schematics that depict the wettability process (contact angle hysteresis) of a planar surface (FIG. 1A) and a surface having an array of microcavities (FIG. 1B) which are used to explain the measures of hydrophilicity and hydrophobicity of solid surfaces and the problematic air entrapment associated with a surface containing microcavities which is addressed by the present disclosure;

FIGS. 2A and 2B are photographs of a water droplet on the traditional ULA coated polystyrene surface (FIG. 2A—PRIOR ART) and a water droplet on an embodiment of the dissolvable carbohydrate coating on a ULA coated polystyrene surface (FIG. 2B);

FIG. 3 is a photograph of a traditional ULA coated polystyrene surface (FIG. 3—PRIOR ART) shown located in front of a black and white poster showing the clarity of the material;

FIG. 4 is a photograph of a dissolvable carbohydrate coated ULA coated polystyrene surface (FIG. 4) configured in accordance of an embodiment of the present disclosure, shown located in front of a black and white poster showing the clarity of the material;

FIG. 5 is a schematic partial side-view of an exemplary device which has a polymer surface with a water dissolvable carbohydrate coating located thereon in accordance with an embodiment of the present disclosure;

FIG. 6 is a schematic partial side-view of an exemplary device which has a polymer surface coated with a low protein binding coating which in turn is coated with a water dissolvable carbohydrate coating in accordance with an embodiment of the present disclosure;

FIGS. 7A-7D are four schematic partial side-views illustrating four different exemplary shapes of the devices shown in FIGS. 5-6 in accordance with different embodiments of the present disclosure. FIGS. 7C and 7D are blown-up images of the area shown in the square at FIG. 7B, illustrating different embodiments of the device;

FIG. 8 is a flowchart illustrating the steps of an exemplary method for manufacturing the devices shown in FIGS. 5-7 in accordance with an embodiment of the present disclosure;

FIG. 9 is a flowchart illustrating the steps of an exemplary method for using the devices shown in FIGS. 5-7 in accordance with an embodiment of the present disclosure; and,

FIGS. 10A and 10B are drawings showing embodiments of an exemplary cell culture device incorporating the dissolvable carbohydrate coating. FIG. 10B is a blown-up view of the area shown in the square in FIG. 10A; and,

FIGS. 11A and 11B are photographs that show different views of 3D cell aggregates of HCT 116 human colorectal carcinoma cells (ATCC® CCL-247™) after 3 days culture in dissolvable carbohydrate/ULA coated polystyrene microcavities with smooth inner surfaces (culture format: 24 well plate) in accordance with an embodiment of the present disclosure.

### DEFINITIONS

For purposes of this disclosure, the term “coating” means a material applied to the surface of another material. For example, a coating may be a polysaccharide applied to a plastic surface. The coating may be a layer or a film, and may be continuous or discontinuous. The coating may be applied by, for example, spray coating, solvent coating or spin coating techniques or any other technique known in the art.

For purposes of this disclosure, the term “microcavity” means a concavity, a dent, a pit, or a depression in a surface, suitably sized to contain cells cultured in 3D or as spheroids. A cell culture device having “microcavities” is a 3D cell culture device. That is, they are structured to support cells growing in culture in three dimensional configuration, or as spheroids. “Microcavity” and “microwell” are synonymous.

For purposes of this disclosure, the term “microcavity array” means an array of microcavities, suitable for containing one or more spheroids in culture. Cell culture devices having an array of microcavities are 3D cell culture devices. That is, they are structured to support cells growing in culture in three dimensions, or as spheroids.

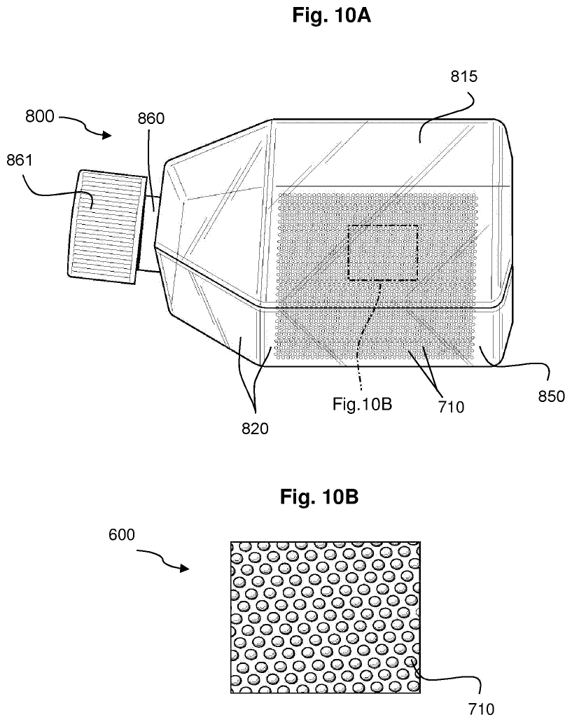

For purposes of this disclosure, the term “3D cell culture device” means a device configured to support cultured cells in forming three dimensional structures such as spheroids. A “3D cell culture device” includes cell culture devices having a microcavity or an array of microcavities (such as the one shown in FIG. 10A). A “3D cell culture device” also includes devices otherwise configured to support cultured cells to form three dimensional structures such as spheroids. For example, a round-bottom multi-well cell culture plate may promote 3D cell growth without a microcavity or a microcavity array.

For purposes of this disclosure, the term “2D cell culture device” means a device have a flat surface for culturing cells, so that cells grow in the cell culture device as two dimensional sheets of cells.

For purposes of this disclosure, the term “microfluidic device” means a device for cell culture having more than one cell culture chamber (such as a well of a multi-well plate or a layer of a multi-layer device” and a mechanism for sharing fluid between the chambers.

For purposes of this disclosure, the term “spheroid” means a group or aggregation of cells growing in a three dimensional configuration in culture. Cells growing in a spheroid configuration are distinguished from cells growing in culture in a two dimensional configuration, as a single layer of cells disposed on a flat cell growth surface.

For purposes of this disclosure, the term “well” means a container for cell culture, which may be a well of a petri dish, a six well plate, a 12 well plate, a 24 well plate, a 96 well plate, a 1536 well plate, a flask, a multi-layer flask, etc.

For purposes of this disclosure, the term “rough” means a surface that is not smooth. For example, flat cell culture surface may be smooth or may deviate from smooth. Surface roughness is quantified by the deviations in the direction of the normal vector of real surface from its ideal form″ (https://en.wikipedia.org/wiki/Surface_roughness). For example, a rough surface may deviate in the direction of the normal vector of a surface by 1 um and less. A surface may be rough or smooth. A microcavity may have one or more internal surfaces which may be rough or smooth.

For purposes of this disclosure, the term “low protein binding surface” or “low binding surface” or “low protein binding coating” or “low binding coating” (all used interchangeably) means a material that has a coating of a material which possesses low protein binding or low cell binding properties. That is, proteins and cells do not bind, or exhibit reduced binding to a low protein binding surface. In embodiments a low protein binding material may be covalently bound to the surface to form a low protein binding surface. In embodiments, the low protein binding surface may be present in a commercially available product such as ULA (Ultra Low Adhesion)-treated cell culture containers available from Corning Incorporated or other similar products. Low protein binding coatings may have hydrophilic characteristics in addition to their low protein binding characteristics.

For purposes of this disclosure, the term “stabilizer” means a water soluble polymer from the polyethylene epoxide family or its biocompatible copolymer derivatives such as polyethylene oxide/polypropylene oxide block copolymers (for example Pluronic F-68, BASF Corporation). In embodiments, this stabilizer may be blended into the carbohydrate coating to help stabilize the dissolvable carbohydrate coating during long term storage. In some embodiments, the stabilizer is polyethylene oxide.

### DETAILED DESCRIPTION

This present disclosure describes various surface modification techniques that can be made to a device (e.g., 3D cell culture device, 2D cell culture device, microfluidic device) to temporarily increase the device's surface hydrophilicity. Although the present disclosure is mainly directed to a 3D cell culture device it should be appreciated that the unique surface modifications described herein can be used with other types of devices such as, for example, 2D cell culture devices and microfluidic devices. As described herein, cell culture devices with various surface geometries (e.g., planar or 2Dsurfaces, or surfaces having at least one microcavity or an array of microcavities, or 3D surfaces) which are modified by the dissolvable hydrophilic coating described herein are biocompatible. In additional embodiments the surfaces further have non-adherent properties. These surfaces having non-adherent properties and microcavities may be used to generate 3D cellular aggregates or spheroids. Further, the disclosed surface modifications also may eliminate air entrapment in the microcavity surfaces during the cell seeding and aqueous media addition steps associated with generating 3D cellular aggregates.

In embodiments, the 2D or 3D cell culture surfaces have a carbohydrate coating. The presence of the carbohydrate coating renders the surface more wettable. That is, the carbohydrate coating is hydrophilic. It attracts water molecules. When the carbohydrate coating is present, the formation of air bubbles upon the introduction of an aqueous liquid onto or into the cell culture surface is decreased. In other words, the carbohydrate coating modifies the hydrophilicity of the surface to prevent air entrapment on the surface of the device when aqueous medium is added to the device. The carbohydrate coating is also biocompatible with cells. Cell culture medium contains carbohydrates such as, for example, glucose or galactose as an energy source for the cells in culture. When these materials dissolve into the media, their presence simply acts to augment the concentration of carbohydrate that already exists in the aqueous media.

In embodiments, the dissolvable carbohydrate coating can be continuous or discontinuous. In embodiments, the dissolvable carbohydrate coating has a thickness limited only by the functionality of the coating. For example the coating may be very thin or may be thick, or may be between thick and thin. The range of thickness of the coating is limited on the thin side by its function—that is, the coating must be thick enough to enable surface wetting, and not so thick as to provide so much glucose to an aqueous media to be out of range for cell culture conditions. For example, the material may range in thickness from thin (i.e. 0.01 micrometers—thick (i.e. 5 micrometers).

A wide range of biocompatible carbohydrates can be used to coat the devices including (for example) monosaccharides, disaccharides, oligosaccharides, and polysaccharides. For example, the devices can be coated with a coating of glucose, galactose, fructose, xylose sucrose, lactose, maltose, trehalose, sorbitol or mannitol. In embodiments, the carbohydrate is provided to a cell culture surface in manner such the coating is not chemically bound to the surface so that the carbohydrate material will be available to dissolve into the media.

Cellulose, for example, is not a suitable carbohydrate for the coating because it is not soluble in water or liquid media (see, for example, Journal of Adhesion Science and Technology 22 (2008) 545-567, Table 1c, which indicates that the value for water adhesion tension (column 3 in table 1) for cellulose film is around 50. This indicates that air bubbles would likely be entrapped inside a microcavity coated with a cellulose coating.)

A more detailed discussion about the devices with the surface coating thereon is described below after a brief discussion is provided to explain the wettability properties of, for example, hydrophilic, superhydrophilic, and hydrophobic surfaces.

In embodiments, the carbohydrate coating may be applied to a cell culture surface having a low protein binding surface treatment. For example, the carbohydrate coating may be applied to a cell culture surface that has been treated (before the application of a carbohydrate coating) with a material which renders the cell culture surface non-adherent to cells. An example of a line of products which have wells with a non-adherent cell culture surface referred to as an ultra low attachment (ULA) surface are sold by Corning Incorporated. The carbohydrate coating may be applied to commercially available ULA treated surfaces.

The carbohydrate coating can be applied by, for example, spray coating, solvent coating or spin coating techniques. In one example, the carbohydrate solution is in a solvent which is compatible with the particular polymer surface material and is applied to the surface such that the solvent is evaporated leaving a thin coating of the carbohydrate. In embodiments, the high affinity of the carbohydrate coating to water molecules yields instantaneous wetting of the surface upon contact with water based solutions such a cell culture media.

In experiments, contact angles of water droplets on embodiments of surfaces having carbohydrate coatings demonstrated that the addition of the carbohydrate coating on a flat ULA-coated polystyrene surface decreased the contact angle from 40° to 10°, rendering the surface superhydrophilic (see TABLE #3).

In additional embodiments, the addition of a water soluble polymer from the polyethyleneoxide family or its biocompatible copolymer derivatives such as polyethylene oxide/polypropylene oxide block copolymers (for example Pluronic F-68, BASF Corporation) to the carbohydrate coating helps to stabilize the uniform coating of resulting carbohydrate coating from around 24 hours to 30-45 days so it is suitable for long term storage at ambient conditions.

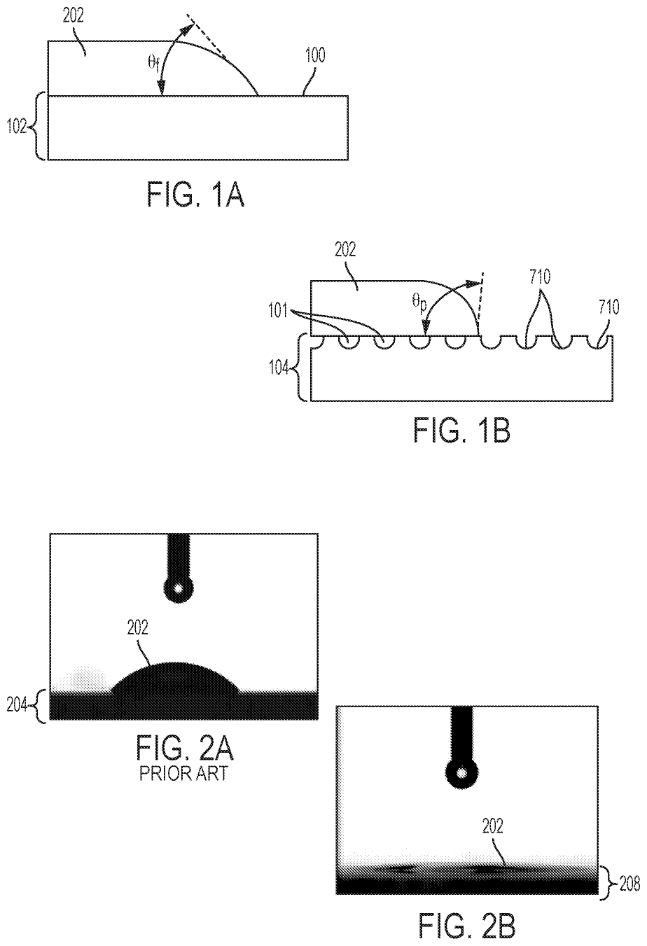

A hydrophilic surface can be generally described as a surface that attracts water and the water contact angle should be less than 90° (e.g., see discussion below and FIGS. 1A-1B, FIGS. 2A and 2B, and TABLES #1-3 for more details about a contact angle and how it relates to other kinds of surfaces in addition to a hydrophilic surface). The energy balance for a hydrophilic surface expressed by Youngs's equation can be written as:

γₛ−γₛ₁=γ₁ cos θ (equation no. 1)

where γₛ is the solid surface free energy, γ₁ is the liquid surface free energy (the liquid surface tension), γₛ₁ is the solid/liquid interfacial free energy, and θ is the equilibrium contact angle. For superhydrophilic surfaces the contact angle is zero which is the limit of applicability of Young's equation. In general, superhydrophilic surfaces are hydrophilic and also have surface roughness.

As such, hydrophilic systems are better characterized by the work of liquid spreading Wₛ− work performed to spread a liquid over a unit surface area of a clean and nonreactive solid as follows:

Wₛ=γₛ−(γ₁+γₛ₁) (equation no. 2)

If the liquid does not spread completely but forms a define contact angle, then the work of spreading can be calculated from measured contact angles and surface tension of liquid as long as θ>0 as follows:

Wₛ=γ₁(cos θ−1) (equation no. 3)

Therefore, the work of spreading could be used as measure of solid surface hydrophilicity. For more details about this work of spreading equation, reference is made to C. J. Van Oss “Interfacial Forces in Aqueous Media”, Marcel Dekke Inc, New York, 1994 (the contents of this document are hereby incorporated by reference herein for all purposes).

To account for the interaction of the liquid with the solid surface during the wetting process, the free energy of hydration/solvation (ΔGₛ₁) can be used as the absolute measure of hydrophilicity. Hydrophobic molecules which attract each other in water have ΔGₛ₁>−133 mJm⁻², whereas for hydrophilic molecules ΔGₛ₁<−133 mJm⁻² (see the aforementioned C. J. Van Oss, Interfacial Forces in Aqueous Media, Marcel Dekke Inc, New York, 1994). Taking this into account, equation no. 3 can then be modified to read as follows:

ΔGₛ₁=−γ₁(cos θ+1) (equation no. 4)

The equilibrium contact angle which describes the transition between hydrophilic and hydrophobic surfaces is θ=56° for ΔGₛ₁=−113 mJ m⁻². Thus, the various measures of hydrophilicity of solid surfaces can be summarized as shown below in TABLE #1 which indicates the commonly accepted measures of hydrophilicity and hydrophobicity of solid surfaces:

TABLE #1

| | | Water adhesion | Energy of |
|---|---|---|---|
| Type of | Contact | tension, | hydration, |
| surfaces | angle, θ | mJ m⁻² | mJ m⁻² |
| Superhydrophilic, | 0° | ≥73 | ≤−146 |
| r ≥ 1 | | | |
| Hydrophilic | 0° | ≥73 | ≤−146 |
| Weakly | (56°-65°) ≥ | 73 to 35 | −146 to −113 |
| hydrophilic | θ ≥ 0° | | |
| Weakly | 90° > θ > | 30-40 to 0 | −73 to −113 |
| hydrophobic | (56-65°) | | |
| Hydrophobic | 120° > θ > 90° | 0 to −36 | −36 to −73 |
| Superhydrophobic | θ > 150° | <−36 | ΔGₛₗ > −10 |

Note 1:
Table is cited from Soft Matter, 2011, 7, 9804-9828.

Note 2: In this table, the surface that is being referred to as a superhydrophilic surface is a textured and/or structured surface (rough and/or porous) possessing a roughness factor (r=ratio of real surface area to projected surface area) larger than 1 (i.e., r>1) on which water (liquid) spreads completely. In other words, a superhydrophilic (superwetting) surface is be achieved by introducing roughness to the hydrophilic materials.

Note 3: FIGS. 1A and 1B are schematics that depict the wetting process for a smooth planar surface 100 (FIG. 1A) and a surface having an array of microcavities 710 (FIG. 1B), one form of a 3D cell culture surface. The surfaces illustrated in FIG. 1A and FIG. 1B may or may not be treated with a surface coating, such as an Ultra Low Attachment (ULA) coating. FIG. 1A shows a droplet of aqueous liquid 202 applied to a smooth flat surface 100 of a polymer material 102. FIG. 1B illustrates a droplet of aqueous liquid 202 applied to a surface of a polymer material 104 having microcavities 710. In the absence of a carbohydrate coating, air 101 may become trapped in the micro cavity 710 features of the surface. To reduce the formation of air bubbles 101, which interfere with cell culture in the microcavities 710, the surface can be treated to enhance its hydrophilicity.

The enhancement of hydrophilicity of surfaces can be approached through either the deposition of a molecular or microscopic coating of a material that is more hydrophilic than the substrate, or by the modification of the chemistry of the substrate itself. Both, molecular modification and modification of surface chemistry have been used in the past in the case of polymeric materials in life science applications.

It is known that a number of organic molecules from either a solution or vapor phase can absorb on selected solids, and organize themselves into self-assembled monolayers which change the wetting characteristics of substrates. For more details about these self-assembled monolayers, reference is made to A. Ulman, “An Introduction to Ultrathin Organic Films: From Langmuir-Blodgett to Self-Assembly”, Academic Press Inc, Boston, 1991 (the contents of this document are hereby incorporated by reference herein for all purposes).

Besides arranging self-assembled monolayers of chemically bonded short functional molecules onto solid surfaces, a great deal of work has been focused on the coating of materials with macromolecules for the modification of polymers used in life sciences applications. However, in the typical bioengineering or cell culture applications, the hydrophilicity of grafted coatings or physically adsorbed synthetic macromolecule coatings is usually of secondary importance since the biocompatibility of the device with the cell media is more important. Instead, the typical protective coatings are intended to prevent protein adsorption when the surface comes in contact with biological fluids. For more details about this type of work, reference is made to D. G. Castner et al. “Biomedical Surface Science: Foundations to Frontiers”, Surf. Sci., 2002, 500 (1-3), 28-60 (the contents of which are hereby incorporated by reference herein for all purposes).

One example of such a protective or a low protein binding coating used in industry today that was developed by Corning Incorporated is the ultra-low attachment (ULA) coating which is applied to a cell culture surface. Products with ULA applied to a polystyrene surface are commercially available from Corning Incorporated. The ultra-low attached surface is a covalently bound hydrogel layer that is hydrophilic and neutrally charged. The main goal of the ULA coating is to minimize the adsorption of the biological molecules from the cell culture media into the surface, thus preventing the cells from attaching to the surface, rendering it a low protein binding surface.

Corning Incorporated has also recently developed new cell culture vessels which have individual microcavities situated within a well of a multi-well plate. For example, a 96 well plate has 96 wells, and within each of these wells, a single spheroid may grow (Spheroid Plate, Corning Incorporated). These products are designed for the generation and culturing of 3D cell aggregates. These cell culture vessels also utilize a ULA coating to prevent cell attachment and could use the new carbohydrate thin coating (e.g., glucose thin film coating) described in the present disclosure. For more details about these new cell culture vessels, reference is made to the co-assigned Patent Application Serial No. PCT/US2015/58048 filed 29 Oct. 2015, and entitled “Devices and Methods for Generation and Culture of 3D Cell Aggregates” (the contents of this document are hereby incorporated by reference herein for all purposes). Again, the main goal of the ULA coating is to prevent the attachment of cells to the cell culture surface in order to promote the formation of 3D aggregates.

The traditional flat ULA coated polystyrene surface demonstrates weakly hydrophilic properties with a contact angle varying from 30° to 50° (as shown in FIG. 1A and Table #2). However, the introduction of microcavities to the ULA coated polystyrene surface increases the apparent contact angle (as shown schematically in FIG. 1B and as measured in Table #2). FIGS. 1A and 1B depict schematics of the wettability of a planar surface 101 (FIG. 1A) compared to a surface containing an array of microcavities 710 (FIG. 1B). FIGS. 1A and 1B depict surfaces made from the same polymer material. In FIGS. 1A and 1B, it can be seen that θₚ>θ_{f} and it should be appreciated that if θₚ≥120° air locking occurs (entrapment of air 101 in the microcavity features 710 of the surface) (note: θ_{f} is contact angle associated with planar surface 102 and θₚ is contact angle associated with microcavity-containing surface 104. In practice the contact angle is generally represented by θ regardless of the type of surface). In the case of a microcavity array surface (a surface having an array of microcavities 710, see, for example, 600 of FIG. 10B). With a roughness factor r>1, air bubbles can be entrapped in the microcavities 710. This system can be described by the Cassie-Baxter model as follows:

cos θ_{C-B}=φₛ cos θ−(1−φₛ) (equation no. 5)

where φₛ is the fraction of the liquid base in contact with the solid surface, φₛ<1, and (1−φ) is the fraction of the liquid base in contact with air pockets. For more details about the Cassie-Baxter model (cos θ_{C-B}), reference is made to A. B. Cassie et al. “Wettablity of Porous Surfaces” Trans. Faraday Soc., 1944, 40, 546-551 (the contents of this document are hereby incorporated by reference herein for all purposes).

An analysis of the measured contact angles (measured according to EXAMPLE 3) of ULA coated flat and microcavity-containing polystyrene surfaces and the amount of entrapped air bubbles thereon to demonstrate how the Cassie-Baxter model describes the wettability of surfaces having an array of microcavities is provided in TABLE #2 as follows:

TABLE #2

| | Contact | Bubble entrapment, |
|---|---|---|
| | angle θ, | % of total liquid |
| Sample | degrees | coated microwells |
| Polystyrene smooth surface | 90 | N/A |
| (uncoated) | | |
| Polystyrene surface containing | 132 | 100 |
| an array of microcavities | | |
| (uncoated) | | |
| ULA coated flat polystyrene | 40° | N/A, φ = 1 |
| surface | | |
| ULA coated polystyrene surface | 48° | 15, φ = 0.94 |
| containing an array of micro- | | |
| cavities | | |

To obtain increased (up to 100%) wettability of microwells or microcavities 710 during the addition of aqueous cell culture media, it would be desirable to further decrease the contact angle of the ULA treated or untreated polystyrene surface. And, at the same time it would be desirable to ensure that the chemical functional groups of the ULA coating remain present when the cell media solution is applied to inhibit cell adhesion to the surface.

In embodiments, a dissolvable carbohydrate coating provides a cell culture surface that is temporarily more hydrophilic to reduce the formation of bubbles upon the introduction of an aqueous liquid to the cell culture surface. In additional embodiments, the combination of a dissolvable carbohydrate coating with a low protein binding surface, such as a polymer surface that has been treated with ULA, provides a surface that (1) increases the wettability of the surface to prevent the formation of air bubbles; and also (2) provides a surface that prevents cell attachment and encourages cells to grow in the form of a spheroid. A detailed discussion about these embodiments are provided to describe a specific embodiment of the present disclosure where the new devices have coating of a dissolvable carbohydrate material.

In embodiments, the coating is thin. The range of thickness of the coating is limited on the thin side by its function—that is, the coating must be thick enough to enable surface wetting, and not so thick as to provide so much glucose to an aqueous media to be out of range for cell culture conditions. For example, the material may range in thickness from thin (i.e. 0.01 micrometers—thick (i.e. 1-2 micrometers or up to 5 micrometers) on top of a ULA coated polystyrene surface. A detailed discussion is provided to describe several general embodiments of devices which are configured in accordance with the present disclosure to have a polymer surface with a water dissolvable carbohydrate coating located thereon that is both biocompatible and has high surface wettability properties (e.g., contact angles in range of 0°-40°).

In embodiments, the coating is temporary. That is, the coating is dissolvable. In embodiments, upon the addition of aqueous media, the coating dissolves into the media, exposing the surface below the coating. The surface below the coating may be a polymer surface, may be a polymer surface treated with a low binding treatment or coating, may be smooth or may be rough, may contain microcavities and the microcavities themselves may have a smooth or rough surface. In embodiments, when the dissolvable coating dissolves into the aqueous media, it does not have an adverse effect on the cells cultured in that media. That is, the dissolvable coating is non-toxic to cells in culture.

In an embodiment of the present disclosure, the cell culture surface has a coating or layer of glucose deposited on top of a ULA coated polystyrene surface (e.g., see FIG. 6). The deposition of glucose on top of a ULA coated polystyrene surface has been found to decrease the contact angle of the surface from 40° to 10° (for flat surfaces) and 48° to 30° (for surfaces having an array of microcavities) (comparing the contact angle measurements for ULA coated flat polystyrene and ULA coated polystyrene having an array of microcavities shown in TABLE #2 with the contact angle measurements for glucose/ULA coated flat and microcavity-containing polystyrene surfaces shown in TABLE #3, below). Glucose, and other carbohydrates, are common ingredients in cell culture media, to provide nutrition to the cells in culture.

FIG. 2A (PRIOR ART) is a photograph that shows a water droplet 202 on a prior art ULA coated polystyrene surface 204 which is exhibiting weakly hydrophilic properties (contact angle in range of (56°-65°)≥θ≥0° per TABLE #1). FIG. 2B is a photograph that shows a water droplet 206 on the dissolvable carbohydrate coated (glucose-coated)—ULA coated polystyrene surface 208 which is exhibiting hydrophilic properties (contact angle 0° per TABLE #1). The dissolvable carbohydrate coated cell culture articles (glucose-ULA coated) demonstrated stronger hydrophilic properties.

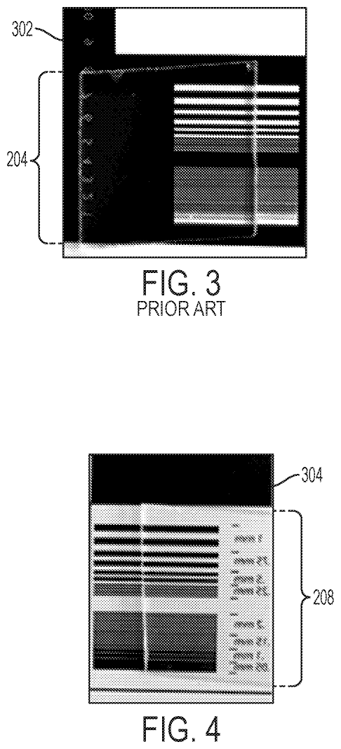

To illustrate that the dissolvable carbohydrate coating does not interfere with the clarity of the polymer surfaces, FIGS. 3 and 4 are provided. FIGS. 3 and 4 are photographs of the traditional ULA coated polystyrene surface 204 (FIG. 3—PRIOR ART) located in front of a black and white poster 302. FIG. 4 shows a ULA polystyrene surface coated with a dissolvable carbohydrate material 208 (in this embodiment, glucose) located in front of a black and white poster 304. FIG. 4 demonstrates that the glucose ULA coated polystyrene surface 208 remained completely transparent after the addition of the dissolvable carbohydrate coating. In particular, FIGS. 3 and 4 show a comparison of the optical transparency of the traditional ULA coated polystyrene surface 204 and the dissolvable carbohydrate coated ULA coated polystyrene surface 208 where the photographs indicate that the glucose coating does not affect the optical transparency of the polystyrene.

Described below are several general embodiments of devices 500, 600, 700 and 800 (e.g., 3D cell culture devices which have an array of microcavities, 2D cell culture devices (having flat surfaces), and microfluidic devices), in accordance with the present disclosure, which have a polymer surface with a dissolvable carbohydrate coating located thereon that is both biocompatible and has high surface wettability properties (e.g., contact angle in a range of 0°-40°).

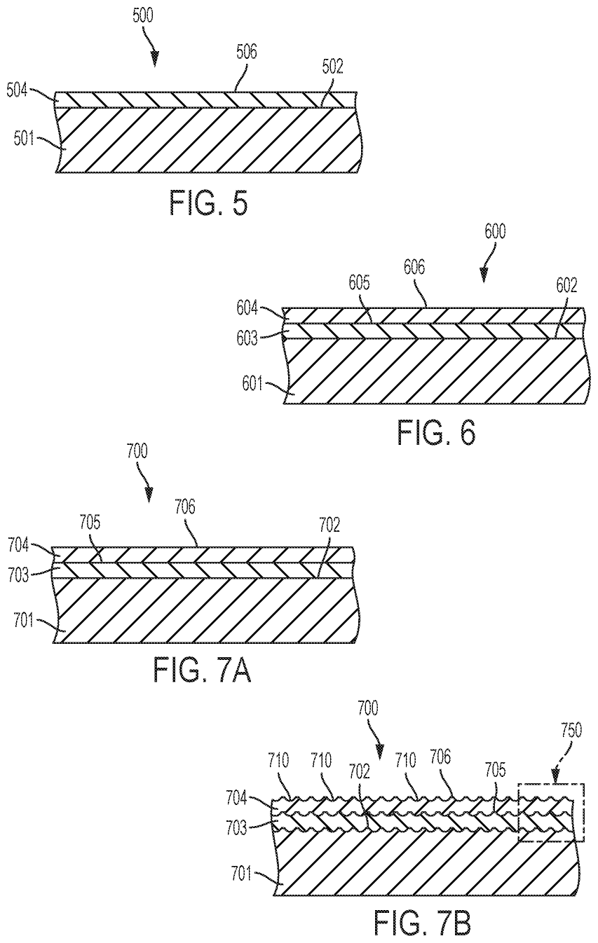

Referring to FIG. 5, there is shown a schematic partial side-view of an exemplary device 500 which has a polymer substrate 501 having a top surface 502 and a dissolvable carbohydrate coating 504, having a top surface 506, in accordance with an embodiment of the present disclosure. The polymer substrate 502 can be any surface suitable for cell culture, and may be one of the following (for example): ethylene vinyl acetate, polypropylene, polyolefin, polystyrene (e.g., plasma-treated polystyrene), polycarbonate, polyester, copolymers of polyester, and a fluoropolymer. In embodiments, the polymer substrate may be porous or non-porous, permeable to small molecules, gas permeable or gas permeable, liquid impermeable. The dissolvable carbohydrate coating 504 can be made from a wide range of biocompatible carbohydrates including (for example) monosaccharides, disaccharides, oligosaccharides, and polysaccharides. For example, the dissolvable carbohydrate coating 504 can be glucose, galactose, fructose, xylose sucrose, lactose, maltose, trehalose, sorbitol, mannitol.

The device 500 is an improvement over the state-of-the-art devices because it provides a surface that is hydrophilic, reducing the formation of bubbles on or near the surface of the cell culture article. The dissolvable carbohydrate coating 504 not only increases the surface wettability properties but also does not impact the physical-chemical properties of the polymer surface 502 because the carbohydrate coating 504 dissolves into the media upon the addition of an aqueous cell culture media.

The dissolvable carbohydrate coating 504 can also contain a water soluble stabilizer polymer, for example a polymer from the polyethyleneoxide family, to stabilize the dissolvable carbohydrate coating 504 for long term storage (e.g., 30-45 days) at ambient conditions. Glucose coated ULA surfaces (both flat and containing microcavities) are stable when stored in dry conditions (hermetically sealed in a plastic bag that contains a desiccant package). If stored at ambient conditions for more than 24 h (room temperature, relative humidity more than 40%) the glucose coating may bead up into small glucose droplets, thus revealing the underlying surface with its uncoated wettability properties. Table #3 shows contact angles measured from glucose/ULA coated materials, both immediately upon formation of the surfaces and after various storage times and conditions, with and without the addition of a stabilizer polymer (polyethylene oxide), in the presence and absence of an array of microcavities, for 24 hours up to 30 days. Table #3 shows that for stability, surfaces coated with a dissolvable carbohydrate coating may be stored long term in a dry environment. Or, in the alternative, the dissolvable carbohydrate coating may contain a stabilizer polymer such as polyethyleneoxide which provides a substrate having a dissolvable carbohydrate coating which may be stored long term (i.e. longer than 30 days) in ambient conditions while retaining its favorable characteristics.

TABLE #3

| | Contact | Bubble entrapment φ, |
|---|---|---|
| | angle θ, | % of total liquid |
| Sample | degrees | coated microwells) |
| Glucose/ULA coated flat polystyrene | 10° | N/A, φ = 1 |
| surface | | |
| Glucose/ULA coated polystyrene | 30° | 0%, φ = 1 |
| surface having an array of micro- | | |
| cavities | | |
| Glucose/ULA coated flat polystyrene | 40° | N/A, φ = 1 |
| surface after 24 h storage at ambient | | |
| conditions | | |
| Glucose/ULA coated flat polystyrene | 10° | N/A, φ = 1 |
| surface stored for 30 days in sealed | | |
| plastic bag with desiccant | | |
| Polyethyleneoxide-Glucose/ULA | 10° | N/A, φ = 1 |
| coated flat polystyrene surface stored | | |
| for 30 days at ambient conditions t | | |
| Glucose/ULA coated polystyrene | 48° | 15%, φ = 0.94 |
| surface having an array of | | |
| microcavities after 24 h storage at | | |
| ambient conditions. | | |
| Glucose/ULA coated polystyrene | 30° | 0%, φ = 1 |
| surface having an array of | | |
| microcavities stored for 30 days in | | |
| sealed plastic bag with desiccant | | |
| Polyethyleneoxide-Glucose/ULA | 30° | 0%, φ = 1 |
| coated polystyrene surface having an | | |
| array of microcavities stored for 30 | | |
| days at ambient conditions | | |

TABLE #3 shows the improved wetting properties of glucose coated ULA polystyrene with flat surfaces and surfaces containing microcavities. It is believed that for other combinations of flat polymers and carbohydrates discussed in detail hereinafter that the contact angle would be in a range of 0° to 70°. Moreover, it is believed that for other combinations of polymers having an array of microcavities and dissolvable carbohydrate coatings, discussed in detail hereinafter, that the contact angle would be in a range of 0° to 60°.

As can be seen with reference to TABLE #2 and TABLE #3, in the case of the glucose ULA coated polystyrene surfaces having an array of microcavities, there was a decrease in the contact angle from 48° without dissolvable carbohydrate coating to 30° with dissolvable carbohydrate coating. This reduction in contact angle completely eliminated the formation of air pockets in the microcavities or microwells during the initial addition of the cell culture media addition to the device.

Referring to FIG. 6, there is shown a partial side-view of an exemplary device 600 which has a polymer substrate 601 having a top surface 602 coated with a low protein binding coating 603, having a top surface 605 which is also coated with a dissolvable carbohydrate coating 604, which forms a top surface 606 in accordance with embodiments of the present disclosure. The polymer substrate 601 can be one of the following (for example): ethylene vinyl acetate, polypropylene, polyolefin, polystyrene or plasma-treated polystyrene, plasma-treated polystyrene, polycarbonate, polyester, copolymers of polyester, and a fluoropolymer. The low protein binding coating 603 can be a covalently bound hydrogel layer 603 (e.g., molecular monolayer or 0.01 to 5 μm thick) which is also referred to herein as the ULA surface 603. The low protein binding coating has a top surface of the low protein binding coating 605. The dissolvable carbohydrate coating 604, having a top surface 606, can be made from a wide range of biocompatible carbohydrates including (for example) monosaccharides, disaccharides, oligosaccharides, and polysaccharides. For example, the dissolvable carbohydrate coating 604 can be glucose, galactose, fructose, xylose sucrose, lactose, maltose, trehalose, sorbitol, mannitol. The dissolvable carbohydrate coating 604 can also contain a water soluble polymer from the polyethyleneoxide family to stabilize the resulting water dissolvable carbohydrate coating 604 for long term storage (e.g., 30-45 days) at ambient conditions. The device 600 is an improvement over the state-of-the-art devices because it has a dissolvable carbohydrate coating 604 which not only increases the surface wettability properties but also does not impact the physical-chemical properties of the low protein binding polymer surface 605 because the carbohydrate thin coating 604 dissolves upon the addition of an aqueous cell culture media.

The dissolvable carbohydrate coating reduces the formation of bubbles on the surface upon introduction of an aqueous cell culture media to the cell culture device. In addition, the dissolvable carbohydrate coating dissolves upon the addition of aqueous cell culture media to the cell culture device. In this way, the surface characteristics introduced by the presence of a dissolvable carbohydrate coating are quickly removed from the surface. The transient presence of a dissolvable carbohydrate coating does not impact the physical-chemical properties of the polymer surface because it rapidly dissolves into the media, leaving behind the surface properties of the substrate. In embodiments, the substrate is coated with a low binding coating, for example a ULA coating, which remains on the polymer surface after the water dissolvable carbohydrate material has dissolved away into the media.

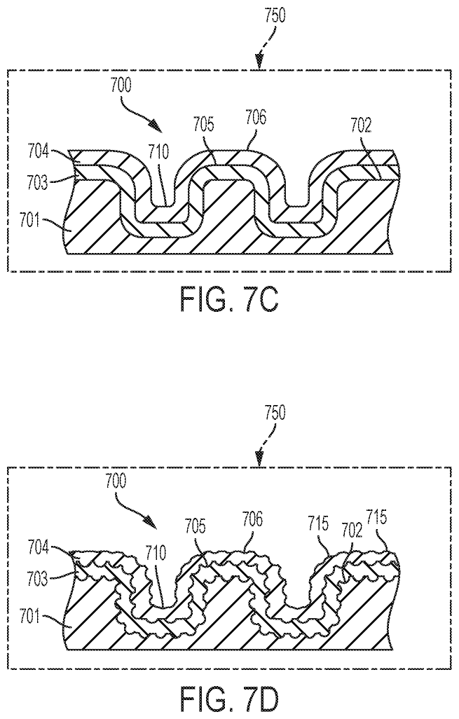

Referring to FIGS. 7A-7D, there are schematic drawings of three partial side-views illustrating four different exemplary shapes of the device 700 in accordance with embodiments of the present disclosure. Shown in FIG. 7A, is a cell culture device 700 having a polymer substrate 701 having a top surface 702. Disposed on the top surface 702 of the polymer substrate 701 is an optional low protein binding coating 703, which, once applied to the top surface 702 of the polymer substrate 701, forms a low protein binding top surface 705. Disposed on top of the optional low protein binding surface coating 703 is a dissolvable carbohydrate coating 704 having a top surface 706. In the embodiment shown in FIG. 7A, the polymer substrate 701 is configured to have a smooth or substantially smooth polymer surface 702 such that the coatings 703 (if present) and 704, provide top surfaces 705 and 706 that are also smooth or substantially smooth. In additional embodiments (not shown), the top surface 702 (705, 706) may be rough.

In FIG. 7B, the top surface 702 of the polymer substrate 701 is configured to have an array of microcavities 710. Because the polymer substrate 701 has an array of microcavities 710 on its top surface 702, the optional low protein binding coating 703 and the dissolvable carbohydrate coating 704 also have an array of microcavities on their top surfaces (705 and 706).

FIGS. 7C and 7D are blown-up views of the area shown at 750 of FIG. 7B. FIGS. 7C and 7D illustrating that the top surface 702 of the polymer substrate 701 is configured to have an array of microcavities 710. Because the polymer substrate 701 has an array of microcavities 710 on its top surface 702, the optional low protein binding coating 703 and the dissolvable carbohydrate coating 704 also have an array of microcavities on their top surfaces (705 and 706). In addition, as shown in FIG. 7D, the microcavities themselves 710 may have a rough surface, as shown by the projections 715 shown in FIG. 7D. While these projections 715 are shown in FIG. 7D as being fairly standardized, or regular, those of ordinary skill in the art will understand that a “rough” surface may have projections or cavities, and that these deviations from “smooth” may be regular or irregular. Reference is made to the definition of the term “rough” above. Also, a flat surface (suitable for 2D cell culture) may be “rough” or “smooth” but still flat as shown in, for example FIGS. 7A and 7B. Similarly, the surface of a microcavity may be “rough” as shown in FIG. 7D or “smooth” as shown in FIG. 7C.

So, as shown in FIG. 7A and FIG. 7B, the top surface of the cell culture article may be smooth (as shown in FIG. 7A) or rough, may contain an array of microcavities which have a smooth surface (as shown in FIGS. 7B and 7C), or may contain an array of microcavities which have a rough surface (as shown in FIG. 7D).

It should be appreciated that these configurations are exemplary and that the devices 500, 600 and 700 can have any desired shape that can be used to enable studies in the molecular, cellular, biological, and other fields. For example, the device may have a shape of a microfluidic device where the inner channels are coated with the dissolvable carbohydrate coating 504, 604 or 704. Further it should be appreciated the devices 500 600 and 700 shown in FIGS. 5-7 and 10 are not to scale where instead in certain embodiments the carbohydrate coating 504, 604 and 704 can be in the range of 0.01-5 μm thick, the hydrophilic coating 603, 703 can be a molecular monolayer or 0.01 to 100 μm, and the polymer surface 502, 602 or 702 can be in the range of 0.01-10 mm (millimeters).

Referring to FIG. 8, a flowchart is provided illustrating an exemplary method 800 for manufacturing the device 500, 600 or 700 (e.g., 3D cell culture device, 2D cell culture device, microfluidic device) in accordance with an embodiment of the present disclosure. The method 800 comprises the steps of (a) providing a polymer substrate 501, 601 or 701, having a top surface 502, 602 or 702, which surface optionally has a low protein binding coating 603, 703 (each having a top surface 605, 705), (step 802), and (b) applying a water dissolvable carbohydrate coating 504, 604 or 704 onto the polymer surface 502, 602 or 702 (step 804). The applying step 804 can include a spray coating step, a solvent coating step, or a spin coating step. For example, the applying step 804 can comprise the steps of bringing (step 804a) a solution of carbohydrate and solvent into direct contact with the polymer surface 502, 602 or 702, and evaporating (step 804b) the solvent from the solution resulting in the polymer surface 502 and 602 being coated with the water dissolvable carbohydrate coating 504, 604, or 704 (optionally in the presence of low protein binding coating which may be covalently bonded to surface 502, 602, or 702 or may not be chemically bound to the polymer surface 502, 602 and 702). The water dissolvable carbohydrate coating 504, 604 and 704 not being chemically bound to the polymer surface 502, 602 and 702 is desirable to allow the dissolvable carbohydrate coating to dissolve away in the presence of aqueous media, leaving behind the native physicochemical properties of the polymer surface 502, 602 and 702, or optionally the physicochemical properties of the low protein binding coating 605 or 705.

In embodiments, the top surface 602, 702 of the polymer substrate 601, 701 may have a low protein binding coating 603, 703 (e.g., covalently bound hydrogel layer 603, 703) on which there is applied the water dissolvable carbohydrate coating 604, 704. The polymer substrate 501, 601 or 701 can be one of the following (for example): ethylene vinyl acetate, polypropylene, polyolefin, polystyrene, plasma-treated polystyrene, polycarbonate, polyester, copolymers of polyester, and a fluoropolymer. The water dissolvable carbohydrate coating 504, 604 or 704 can be made from one of the following (for example) monosaccharides, disaccharides, oligosaccharides, and polysaccharides. The water dissolvable carbohydrate coating 504, 604 or 704 can also contain polyethyleneoxide to stabilize the resulting water dissolvable carbohydrate coating 504, 604 or 704 for long term storage at ambient conditions. The device 500, 600 or 700 is a marked improvement over the state-of-the-art devices because it has a water dissolvable carbohydrate coating 504, 604 or 704 which not only increases the surface wettability properties but also does not impact the physical-chemical properties of the polymer surface 502, 602 and 702 because the dissolvable carbohydrate coating 504, 604 or 704 dissolves upon the addition of a water based cell culture media.

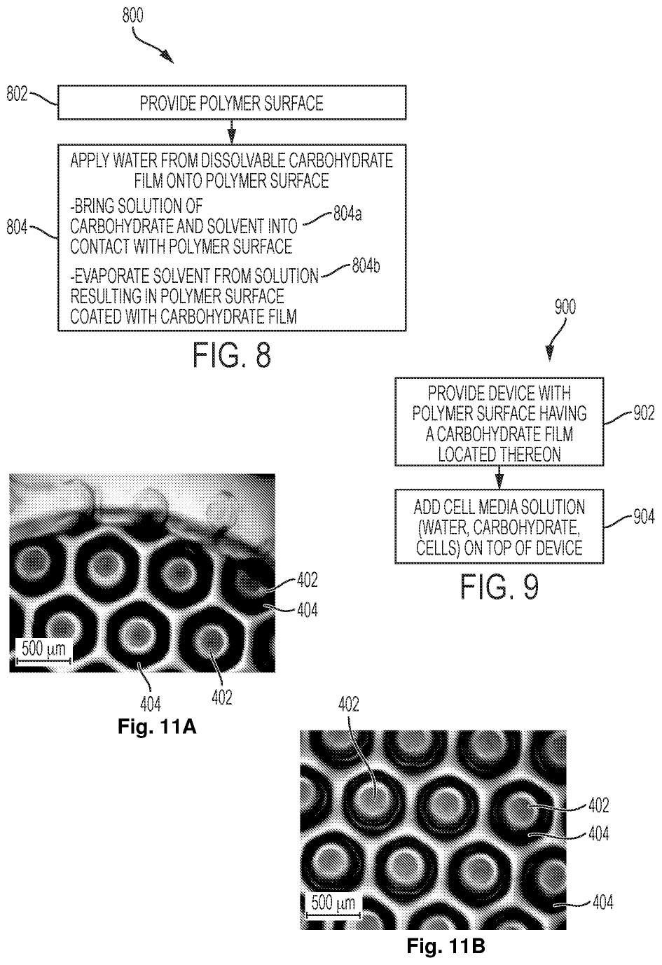

Referring to FIG. 9, there is a flowchart illustrating an exemplary method 900 for using the device 500, 600 and 700, in a form suitable for cell growth (e.g., 3D cell culture device, 2D cell culture device, microfluidic device) in accordance with an embodiment of the present disclosure. The method 900 comprises the steps of (a) providing the device 500, 600 or 700 which has a polymer surface 502, 602 or 702, optionally in the presence of a low protein binding coating (603, 703) with a water dissolvable carbohydrate coating 504, 604 or 704 located thereon (step 902), and (b) adding an aqueous cell culture media solution which contains at least water, a carbohydrate, and cells onto the top surface 506, 606 or 706 of the water dissolvable carbohydrate coating 504, 604 or 704 which is located on the polymer surface 502, 602 or 702 (step 904) or optionally on a low protein binding coating 603 or 703. In embodiments, the polymer surface 602 or 702 may have a low protein binding coating 603 or 703 (e.g., covalently bound hydrogel layer 603 or 703) on which there is present the water dissolvable carbohydrate coating 604 or 704. The polymer surface 502 602 or 702 can be one of the following (for example): ethylene vinyl acetate, polypropylene, polyolefin, polystyrene (e.g., plasma-treated polystyrene), polycarbonate, polyester, copolymers of polyester, and a fluoropolymer. The water dissolvable carbohydrate coating 504, 604 or 704 can be made from one of the following (for example) monosaccharides, disaccharides, oligosaccharides, and polysaccharides. The water dissolvable carbohydrate coating 504, 604 or 704 can also contain polyethyleneoxide to stabilize the resulting water dissolvable carbohydrate coating 504, 604 or 704 for long term storage at ambient conditions. The device 500, 600 or 700 is a marked improvement over the state-of-the-art devices because it has a water dissolvable carbohydrate thin coating 504 and 604 coating which not only increases the surface wettability properties but also, since it dissolves into aqueous solution, does not impact the physical-chemical properties of the polymer surface 502, 602 or 702 or optionally the hydrophilic layer 603 or 703, because the carbohydrate thin coating 504, 604 or 704 dissolves upon the addition of a water based cell culture media. In embodiments, once cell media is added to a cell culture surface having a dissolvable carbohydrate coating, the carbohydrate coating dissolves into the media, leaving behind a polymer surface (with or without an optional low protein binding coating).

Referring to FIGS. 10A and 10B, shown is an embodiment of a cell culture device, in this case a cell culture flask 800 having an array of microcavities 710 on a cell culture surface. The flask 800 has a top wall 815, a bottom wall 850 containing the array of microcavities 710, sidewalls 820, and a removable cap 861, attached to a neck 860 of the flask. FIG. 10B is a blown-up view of the area shown in the square in FIG. 10A, illustrating an array 600 of microcavities 710. In embodiments, the microcavities may be round (as shown in FIGS. 10A and 10B), or hexagonal as shown, for example in FIGS. 11A and 11B, or any other shape. While a cell culture flask is shown, it is to be understood that the cell culture device can be any device suitable for cell culture, and includes a dish, a plate, a well, a multi-well plate (which can have 2, 3, 4, 6, 12, 24, 96 or 1536 wells, or any other number of wells), a flask, a multi-layer flask, or any other suitable cell culture device. These devices containing an array of microcavities are 3D cell culture devices, in embodiments. Devices that are structured to promote 3D cell formation in culture, including 96 well plates having round bottoms (without microcavities) are also 3D cell culture devices.

In an aspect (1) the disclosure provides A device for cell culture comprising a polymer substrate having a top surface; wherein a dissolvable carbohydrate coating is disposed on the top surface of the polymer substrate. In an aspect (2) the disclosure provides the device of aspect 1, wherein the dissolvable carbohydrate coating is selected from the group consisting of: monosaccharides, disaccharides, oligosaccharides, polysaccharides, and mixtures. In an aspect (3) the dissolvable carbohydrate coating further comprises a stabilizer. In an aspect (4) the stabilizer comprises polyethylene oxide. In an aspect (5) the disclosure provides the device of any one of aspects 1-4, wherein the polymer substrate is selected from the group consisting of: ethylene vinyl acetate, polypropylene, polyolefin, polystyrene, polycarbonate, polyester, copolymers of polyester, and a fluoropolymer. In an aspect (6) the polymer substrate is gas permeable, liquid impermeable. In an aspect (7) the disclosure provides the device of any one of aspects 1-6, wherein the polymer substrate comprises a low protein binding coating. In an aspect (8) the disclosure provides the device of any one of claims 1-7, wherein the low protein binding coating comprises a hydrogel layer covalently bound to the polymer top surface. In an aspect (9) the disclosure provides the device of any one of aspects 1-8, wherein the top surface of the polymer substrate comprises a smooth surface. In an aspect (10) the disclosure provides device of any one of aspects 1-9, wherein the top surface of the polymer substrate comprises a rough surface. In an aspect (11) the disclosure provides the device of any one of aspects 1-10, wherein the top surface of the polymer substrate is superhydrophilic. In an aspect (12) the disclosure provides the device of any one of aspects 1-11, wherein the device is selected from the group consisting of a 3D cell culture device, a 2D cell culture device, and a microfluidic device. In an aspect (13) the disclosure provides the device of any one of aspects 1-11 wherein the device is a 3D cell culture device comprising an array of microcavities.

In an aspect (14) the disclosure provides method for manufacturing a device, the method comprising: providing a polymer substrate; and applying a dissolvable carbohydrate coating onto the top surface of the polymer substrate. In an aspect (15) the disclosure provides the method of aspect 14, wherein the applying the dissolvable carbohydrate coating onto the polymer surface comprises: bringing a solution of carbohydrate and solvent into direct contact with the top surface of the polymer substrate; and evaporating the solvent from the solution resulting in the top surface of the polymer substrate comprising dissolvable carbohydrate coating that is not chemically bound to the polymer substrate. In an aspect (16) the disclosure provides the method of aspect 14, wherein the applying step comprises a step selected from the group consisting of a spray coating step, a solvent coating step, and a spin coating step. In an aspect (17) the disclosure provides the method of aspect 14 or 15, wherein, the polymer substrate comprises a low protein binding coating. In an aspect (18) the disclosure provides the method of aspect 17, wherein the hydrophilic coating is a covalently bound hydrogel layer. In an aspect (19) the disclosure provides method of aspect 14, wherein the polymer substrate is selected from the group consisting of ethylene vinyl acetate, polypropylene, polyolefin, polystyrene, polycarbonate, polyester, copolymers of polyester, and a fluoropolymer. In an aspect (20) the disclosure provides the method of aspect 14, wherein the dissolvable carbohydrate coating is selected from the group consisting of monosaccharides, disaccharides, oligosaccharides, polysaccharides, and mixtures. In an aspect (21) the disclosure provides the method of aspect 20, wherein the dissolvable carbohydrate coating further contains polyethyleneoxide.

In another aspect (22), the disclosure method for cell culture comprising:

providing a cell culture device of any one of aspects 1-13; adding a cell culture media solution comprising water, a carbohydrate, and cells to the device.

The following is a more detailed discussion of an experimental process involving the coating of glucose on top of a 24 multi-well plate and the seeding and culturing of 3D cell aggregates. The 24 well plate had a ULA coated smooth polystyrene surface with a microwell pattern prior to the application of the glucose coating which was performed as follows:

### EXAMPLES

### Example 1: Coating

To coat the microwells, 20 μl/cm² of 1% w/v glucose solution in methanol was added into each microwell or microcavity of a 24 well plate. In each case the 24 well plate may be made from uncoated polymer material, such as polystyrene, or may be provided with a low protein binding solution, for example ULA coating from Corning Incorporated. The cell culture surface of the 24 well plates contained microwells or microcavities, but the microwells or microcavities did not themselves have rough interior surfaces. That is, the cell culture surfaces contained microcavities, and the microcavities had smooth surfaces. Methanol was evaporated at 45° C. for 15 min. The glucose coating is equivalent to addition of glucose in the amount of 0.2 mg/cm².

### Example 2: Coatings Using Polyethyleneoxide

It should be noted that due to the hydroscopic nature of glucose this type of coating demonstrates a moderate sensitivity to ambient humidity, e.g., after 24 hour storage in ambient conditions the glucose coating starts to bead up into microscopic droplets on the ULA coated polystyrene surface. To address this issue, the dissolvable carbohydrate coating (in this example, glucose) can be stabilized by the addition of polyethylene oxide (PolyOx WSRN12K, DOW Chemical) into the spray-coating solution used to apply the glucose coating to the ULA coated polystyrene surface. For instance, stable glucose coatings were obtained by spray-coating ULA treated polystyrene surface with 0.25% w/v glucose, 0.1% w/v PolyOx in methanol solution (20 μl/cm²). Then, the residual methanol solvent was dried at 45° C. for 15 min. The glucose-polyethylene oxide coating on the ULA treated polystyrene remained stable at ambient conditions (50% relative humidity, 25° C. room temperature) for at least four days (see Table #3).

### Example 3: Measurement of Contact Angles

The contact angles of all material samples were measured with Kruss DSA30 Drop shape analysis system (Kruss GmbH, Germany). 5 um of distilled water was deposited at 100 ul/min rate at the surface and contact angle was measured by default curve fitting of the droplet shape.

### Example 4: Cell Culture

50 μl/cm² of McCoy's 5A media (Gibco Cat #16600-082) was added into each well of a multi-well plate according to Example 1 and Example 2 to fill the microwells. This step was followed by the addition of 150 μl/cm² of Colon cancer HCT116 cells in suspension in McCoy's media at a concentration of 1 million cells/ml. Cells were allowed to settle into the microwells and the 24 well plate was incubated in a cell culture incubator at 37° C., 5% CO₂, 95% relative humidity.

Images of the cellular spheroids 402 which formed in the glucose ULA coated polystyrene microwells 404 after 3 days incubation are shown in FIGS. 10A and 10B. The glucose coating of the surface increased the overall glucose concentration in the cell culture media during the cell seeding procedure by 20%. However, the glucose concentration returned back to a normal concentration as commonly seen with McCoy's 5A media after subsequent daily cell media exchanges. In general, mammalian cells have a very wide range of tolerance of glucose levels ranging from 1 g/1 to 10 g/L. In the above example, the glucose concentration was raised from 2.95 g/L to 3.5 g/L during the initial cell seeding, which is well within the tolerance range.

The tested glucose-polyethylene oxide coated ULA polystyrene surfaces were completely biocompatible when used in cell studies since the cell culture media contained glucose as a main nutrient at 1-10 g/L concentrations. That is, cells grew in the presence of the glucose polyethylene oxide coated ULA coated polystyrene surfaces. For example, FIGS. 11A and 11B are photographs that show 3D cell aggregates of HCT 116 human colorectal carcinoma cells (ATCC® CCL-247™) (HCT) 116 cells 402 after 3 days culture inside the glucose coated ULA coated microwells 404 with smooth inner surfaces where the glucose coating eliminated air bubble entrapment in the coated smooth polystyrene microwells 404 (culture format: 24 well plate). The 3D spheroids 402 formed inside the ULA coated smooth microwells 404 that were glucose coated prior to cell seeding. No air bubble entrapment was seen inside the glucose coated ULA coated smooth microwells 404 during the addition of the cell culture media.

In view of the foregoing, one skilled in the art will readily appreciate that the present disclosure discloses a device (e.g., 3D cell culture device, 2D cell culture device, microfluidic device) which has a biocompatible dissolvable carbohydrate surface coating with high surface wettability properties. The biocompatible dissolvable carbohydrate surface coating is a water dissolvable carbohydrate coating (e.g., glucose thin film) which effectively converts a hydrophobic or weakly hydrophilic surface into a more hydrophilic surface and significantly reduces the original contact angle of the surface up to 0-10°. Because of the subsequent dissolution of the carbohydrate coating (e.g., glucose thin film) in water based solutions this type of coating does not permanently modify the physical-chemical properties of the original surface. Such dissolvable carbohydrate coatings (e.g., glucose coatings) will be beneficial for modification of surface wettability properties in cell culture applications. Such carbohydrate coatings (e.g., glucose coatings) will especially be useful in the surface modification of surfaces containing an array of microcavities, suitable for providing an environment which encourage the growth of non-adherent spheroids, as opposed to flat, two dimensional cells in culture, to eliminate the entrapment of air in microcavities during the introduction of aqueous media to the surface having microcavities This type of surface coating is completely biocompatible since the carbohydrate (e.g., glucose) is a nutrient provided in cell culture media.

Further, it should be appreciated that the microcavities discussed herein can be any size or shape suitable for the culture of spheroids. For example, in an embodiment, each microcavity may have a closed hemispherical round bottom, an open rounded top having a diameter Dtop, and side walls of increasing diameter between the bottom and the top and having a diameter Dhalf-way between the bottom and the top and a height H above the bottom, wherein Dtop=1.5 to 2.5 Dhalf-way, wherein H=0.7 to 1.3 Dhalf-way, and wherein Dhalf-way is 200 to 1000 μm. And, surfaces containing an array of microcavities discussed herein can be defined, for example, as a planar surface with uniformly packed microcavity or microwell structures in in one of the 11 known uniform tilings of the plane (for example: triangular, square, hexagonal packing) [e.g., see Williams, Robert (1979). The Geometrical Foundation of Natural Structure: A Source Book of Design. Dover Publications, Inc. p. 35-39. ISBN 0-486-23729-X. The contents of this document are incorporated herein by reference for all purposes].

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A device comprising:
a polymer substrate having a cell culture surface;
a dissolvable carbohydrate coating disposed on the cell culture surface of the polymer substrate;
wherein upon the addition of aqueous cell culture media to the coated cell culture surface, the dissolvable carbohydrate coating dissolves into the aqueous cell culture media; and,
wherein the polymer substrate having a dissolvable carbohydrate coating has a contact angle less than the contact angle of the polymer substrate in the absence of the dissolvable carbohydrate coating.

2. The device of claim 1, wherein the dissolvable carbohydrate coating is selected from the group consisting of: monosaccharides, disaccharides, oligosaccharides, polysaccharides, and mixtures.

3. The device of claim 1, wherein the dissolvable carbohydrate coating further comprises a stabilizer.

4. The device of claim 3, wherein the stabilizer comprises polyethylene oxide.

5. The device of claim 1, wherein the polymer substrate is selected from the group consisting of: ethylene vinyl acetate, polypropylene, polyolefin, polystyrene, polycarbonate, polyester, copolymers of polyester, and a fluoropolymer.

6. The device of claim 5, wherein the polymer substrate is gas permeable, liquid impermeable.

7. The device of claim 1, wherein the polymer substrate comprises a low protein binding coating.

8. The device of claim 7, wherein the low protein binding coating comprises a hydrogel layer covalently bound to the polymer top surface.

9. The device of claim 1, wherein the dissolvable carbohydrate coating comprises a stabilizer.

10. The device of claim 9, wherein the stabilizer comprises polyethylene oxide.

11. The device of claim 1, wherein the cell culture surface of the polymer substrate comprises a smooth surface.

12. The device of claim 1, wherein the cell culture surface of the polymer substrate comprises a rough surface.

13. The device of claim 12, wherein the cell culture surface of the polymer substrate is superhydrophilic.

14. The device of claim 1, wherein the device is selected from the group consisting of a 3D cell culture device, a 2D cell culture device, and a microfluidic device.

15. A method for manufacturing a cell culture device having a cell culture surface, the method comprising:
applying a dissolvable carbohydrate coating onto the cell culture surface of the polymer substrate.

16. The method of claim 15, wherein the applying the dissolvable carbohydrate coating onto the polymer surface comprises:
bringing a solution of carbohydrate and solvent into direct contact with the cell culture surface of the polymer substrate; and
evaporating the solvent from the solution resulting in the cell culture surface of the polymer substrate comprising dissolvable carbohydrate coating that is not chemically bound to the polymer substrate.

17. The method of claim 15, wherein the applying step comprises a step selected from the group consisting of a spray coating step, a solvent coating step, and a spin coating step.

18. The method of claim 15, wherein, the polymer substrate comprises a low protein binding coating.

19. The method of claim 18, wherein the hydrophilic coating is a covalently bound hydrogel layer.

20. The method of claim 15, wherein the polymer substrate is selected from the group consisting of ethylene vinyl acetate, polypropylene, polyolefin, polystyrene, polycarbonate, polyester, copolymers of polyester, and a fluoropolymer.

21. The method of claim 15, wherein the dissolvable carbohydrate coating is selected from the group consisting of monosaccharides, disaccharides, oligosaccharides, polysaccharides, and mixtures.

22. The method of claim 15, wherein the dissolvable carbohydrate coating further contains polyethyleneoxide.

23. A method for cell culture comprising:
adding a cell media solution which contains at least water, a carbohydrate, and cells to a device having a cell culture surface comprising a dissolvable carbohydrate coating located thereon; and,
culturing cells.

24. A method for cell culture, comprising: adding a media solution which contains at least water, a carbohydrate, and cells onto a coated cell culture substrate having a cell culture surface comprising a low protein binding coating and a water dissolvable carbohydrate coating on top of the low protein binding coating, and;
culturing cells.

25. The method of claim 24, wherein:
the polymer substrate is selected from the group consisting of ethylene vinyl acetate, polypropylene, polyolefin, polystyrene, polycarbonate, polyester, copolymers of polyester, and a fluoropolymer; and,
wherein the dissolvable carbohydrate coating is selected from the group consisting of: monosaccharides, disaccharides, oligosaccharides, and polysaccharides, or mixtures.
